(19) United States
(12) Patent Application Publication
(10) Pub. No.: US 2026/0118156 A1
(43) Pub. Date: Apr. 30, 2026

(54) FLUID MANAGEMENT SYSTEM WITH ENHANCED LOAD CELL ACCURACY

(71) Applicant: BOSTON SCIENTIFIC SCIMED, INC., Maple Grove, MN (US)

(72) Inventors: ALI ZIEGLER, Lakeville, MA (US); RENELSON MANGALIMAN ALVAREZ, Kilbarry (IE); FEARGAL CLEARY, Trim (IE); LAKSHMI NATARAJAN, Tipperary (IE); CHRISTOPHER NORRIS, Waterford (IE)

(73) Assignee: BOSTON SCIENTIFIC SCIMED, INC., Maple Grove, MN (US)

(21) Appl. No.: 19/372,837

(22) Filed: Oct. 29, 2025

Related U.S. Application Data
(60) Provisional application No. 63/713,784, filed on Oct. 30, 2024.

(51) Int. Cl. G01G 19/18; A61B 1/015; G01G 17/06; G01G 23/01

(52) U.S. Cl. CPC G01G 19/18; A61B 1/015; G01G 17/06; G01G 23/01

(57) ABSTRACT

A fluid management system may include a fluid bag and a measurement load cell for monitoring the state of the fluid bag. The measurement load cell may receive an input voltage and generate a differential voltage across output nodes. A compensation circuit may be coupled to the measurement load cell and include inputs with series resistors, an operational amplifier, and a digital-to-analog converter (DAC) coupled by a current limiting resistor. The system may include a controller configured to measure an actual offset voltage of the measurement load cell during a calibration phase, determine required compensation to bring the offset into a desired range, generate a compensation voltage using the DAC, and apply the compensation voltage to the measurement load cell circuit. This compensation may minimize offsets in the load cell, improving overall system dynamic range and performance for more accurate fluid weight measurements

### CROSS-REFERENCE TO RELATED APPLICATIONS

The application claims the benefit of U.S. Provisional Patent Application Ser. No. 63/713,784, filed on Oct. 30, 2024, the disclosure of which is incorporated herein by reference.

### TECHNICAL FIELD

The disclosure is directed to a fluid management system. More particularly, the disclosure is directed to load cell compensation circuits for improved accuracy in fluid management systems.

### BACKGROUND

Flexible ureteroscopy (fURS), gynecology, and other endoscopic procedures require the circulation of fluid for several reasons. Fluid management systems may be used to deliver fluid to an anatomical cite from a reservoir at a desired pressure and/or flow rate via a peristaltic or roller pump. Fluid management systems may adjust the flow rate and/or pressure at which fluid is delivered from the reservoir based on data collected from a procedural device, such as, but not limited to, pressure readings sensed and/or obtained by the fluid management system. The fluid management system may utilize a disposable fluid tubing set installed with a pump console to provide the fluid to the patient. There is an ongoing need to provide alternative configurations of the components of fluid management systems, to facilitate the use thereof.

### BRIEF SUMMARY

This disclosure provides design, material, manufacturing method, and use alternatives for components of a fluid management system.

In an example, a fluid management system may include a fluid bag and a measurement load cell for monitoring the state of the fluid bag. The measurement load cell may receive an input voltage and generate a differential voltage across a first measurement load cell output node and a second measurement load cell output node. The system may also include a compensation circuit coupled to the measurement load cell that may have a first input with a first series resistor leading to a first input line, a second input with a second series resistor leading to a second input line, a first operational amplifier coupled to the first and second input lines, and a digital to analog converter (DAC) coupled by a current limiting resistor to the second input line. The system may further include a microcontroller electrically coupled to the measurement load cell and the DAC.

Alternatively or additionally to any of the examples above, in another example, the DAC may be configured by the microcontroller to generate a controlled output, and the controlled output is calibrated to the measurement load cell.

Alternatively or additionally to any of the examples above, in another example, the controlled output may be calibrated to the measurement load cell by providing an input voltage to the measurement load cell, monitoring an output of the compensation circuit, and adjusting the DAC until the output of the compensation circuit is in a desired range, and storing a setting for the DAC at which the output of the compensation circuit is in the desired range.

Alternatively or additionally to any of the examples above, in another example, a resistance of the first series resistor and a resistance of the second series resistor may be approximately equal.

Alternatively or additionally to any of the examples above, in another example, a resistance of the current limiting resistor may be at least 10 times the resistance of the first series resistor.

Alternatively or additionally to any of the examples above, in another example, the system may further include a second operational amplifier configured to receive an output voltage from the first operational amplifier.

Alternatively or additionally to any of the examples above, in another example, the system may include a fourth resistor and a fifth resistor coupled to the first operational amplifier and configured to set a gain of the first operational amplifier.

In an example, a fluid management system may include a fluid bag and a measurement load cell for monitoring the state of the fluid bag. The measurement load cell may receive an input voltage and generate a differential voltage across a first measurement load cell output node and a second measurement load cell output node. The system may also include a compensation circuit coupled to the measurement load cell that may have a first input with a first series resistor leading to a first input line, a second input with a second series resistor leading to a second input line, a first operational amplifier coupled to the first and second input lines, and a digital to analog converter (DAC) coupled by a current limiting resistor to the second input line. The system may further include a controller that may be configured to measure an actual offset voltage of the measurement load cell during a calibration phase, determine a required compensation to bring the offset of the measurement load cell into a desired range, generate a compensation voltage using the DAC, and apply the compensation voltage to the circuit of the measurement load cell.

Alternatively or additionally to any of the examples above, in another example, the controller may be further configured to repeat the measuring and determining steps at predetermined intervals to thereby adjust the compensation voltage and account for drift in the characteristics of the measurement load cell.

Alternatively or additionally to any of the examples above, in another example, the desired range may be from about −1 mV/V to about 1 mV/V.

Alternatively or additionally to any of the examples above, in another example, a resistance of the first series resistor and a resistance of the second series resistor may be approximately equal.

Alternatively or additionally to any of the examples above, in another example, a resistance of the current limiting resistor may be at least 10 times the resistance of the first series resistor.

Alternatively or additionally to any of the examples above, in another example, the system may further include a second operational amplifier configured to receive an output voltage from the first operational amplifier.

Alternatively or additionally to any of the examples above, in another example, the system may include a third resistor and a fourth resistor coupled to the first operational amplifier and configured to set a gain of the first operational amplifier.

Alternatively or additionally to any of the examples above, in another example, the gain may be approximately 125.

Alternatively or additionally to any of the examples above, in another example, the system may include a plurality of capacitors configured to filter high-frequency noise from the load cell input signals, stabilize power supplies for the operational amplifiers, smooth the output signal, and stabilize the compensation voltage.

Alternatively or additionally to any of the examples above, in another example, the system may include a reference voltage source coupled to the first operational amplifier.

Alternatively or additionally to any of the examples above, in another example, the DAC may be configured by the controller to generate a controlled output, and the controlled output is calibrated to the measurement load cell.

Alternatively or additionally to any of the examples above, in another example, the controlled output may be calibrated to the measurement load cell by providing an input voltage to the measurement load cell, monitoring an output of the compensation circuit, and adjusting the DAC until the output of the compensation circuit is in the desired range, and storing a setting for the DAC at which the output of the compensation circuit is in the desired range.

In an example, a method for compensating a measurement load cell in a fluid management system may include measuring an actual offset voltage of the measurement load cell during a calibration phase, determining a required compensation to bring the offset of the measurement load cell into a desired range, generating a compensation voltage using a digital to analog converter (DAC), applying the compensation voltage to the circuit of the measurement load cell, and amplifying the compensated measurement load cell signal using a second operational amplifier with a gain of approximately 125.

Alternatively or additionally to any of the examples above, in another example, the method may include repeating the measuring and determining steps at predetermined intervals to thereby adjust the compensation voltage and account for drift in the characteristics of the measurement load cell.

Alternatively or additionally to any of the examples above, in another example, the desired range may be from about-1 mV/V to about 1 mV/V.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is an enlarged view of a portion of the illustrative console of FIG. 1;

FIG. 3 is a circuit diagram of an illustrative compensation circuit; and

FIG. 4 is a flow diagram of an illustrative method for determining an actual offset of a load cell and applying a corrective bias.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may control the flow of fluid into the body cavity and/or regulate body cavity pressure and/or the flow rate of fluid flow to the body cavity using an inflow and/or outflow pump of the fluid management system. The inflow pump may deliver fluid through inflow tubing of a fluid tubing set to the patient and/or the outflow pump may remove fluid through outflow tubing of a fluid tubing set from the patient. The fluid management system may include one or more load cells which measure a hung saline bag weight. The weight of the hung saline bag may be displayed on a display screen to inform the physician how much saline they have left before they must change the saline bag. It is important that the amount displayed on the screen is accurate so the physician can accurately predict when they need to switch out the bag and not waste precious time during a procedure. However, the manufacturing variability in load cells may cause each load cell to have an offset of varying magnitude. This inherent offset may negatively impact the measurement system because it limits the workable gain and decreases resolution. The present disclosure is directed towards systems and methods for minimizing the offset to improve the overall system dynamic range and performance and output a higher accuracy signal from the load cell.

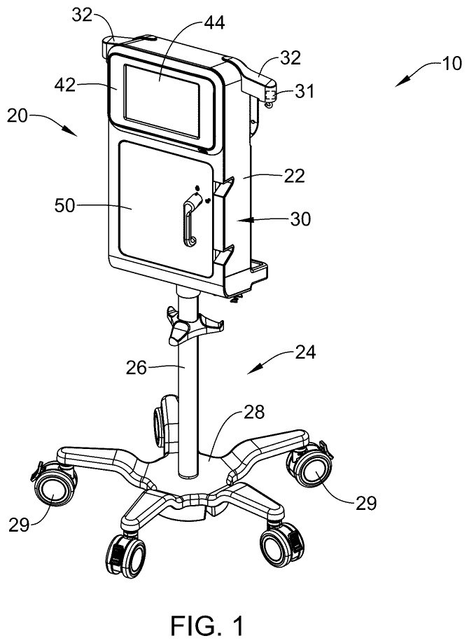

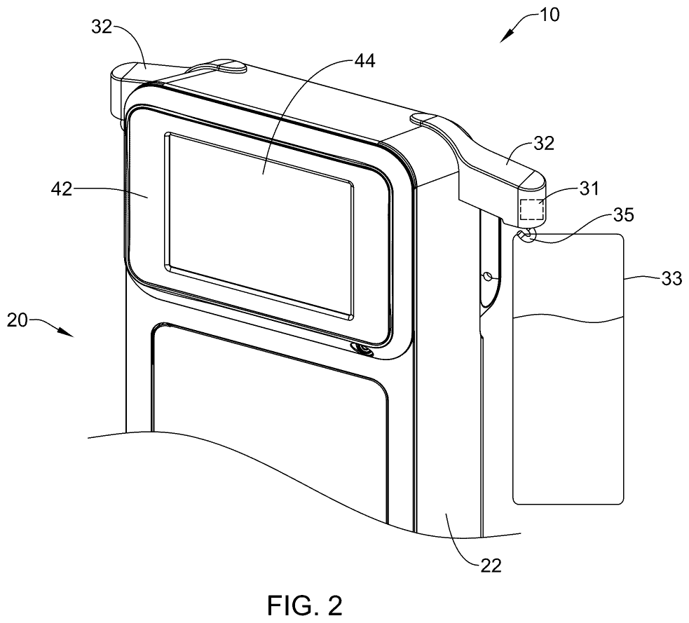

FIG. 1 is a schematic view of a fluid management system 10 that may be used in an endoscopic procedure, such as fURS procedures. FIG. 2 is an enlarged view of a portion of the fluid management system 10 of FIG. 1. The fluid management system 10 may be coupled to a medical device (not shown), such as an endoscope, that allows a flow of fluid therethrough. The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. For example, the wheeled cart 24 may include a pole 26 extending upward from a base 28. The base 28 may include a plurality of wheels 29 (e.g., caster wheels), allowing the cart 24 to be wheeled around to a desired location. In other instances, the console 20 may be provided with another form of cart, configured to be positioned on a flat surface, mounted to a wall, etc.

The fluid management system 10 may also include one or more user interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The touch screen interface 42 allows the user to input/adjust various functions of the fluid management system 10 such as, for example, flow rate, pressure, and/or temperature. The user may also configure parameters and alarms (such as, but not limited to, a max pressure alarm), information to be displayed, and the procedure mode. The touch screen interface 42 allows the user to add, change, and/or discontinue the use of various modular systems within the fluid management system 10. The touch screen interface 42 may also be used to change the fluid management system 10 between automatic and manual modes for various procedures. It is contemplated that other systems configured to receive user input may be used in place of or in addition to the touch screen interface 42 such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flow rate and/or fluid pressure display. In some embodiments, operating parameters may be adjusted by touching a corresponding portion of the touch screen interface 42. The touch screen interface 42 may also display visual alerts and/or audio alarms if parameters (e.g., flow rate, temperature, etc.) are above or below predetermined thresholds and/or ranges. In some embodiments, the fluid management system 10 may also include further user interface components such as an optional foot pedal, a fluid warmer user interface, a fluid control interface, or other device to manually control various modular systems. For example, an optional foot pedal may be used to manually control flow rate. Some illustrative display screens 44 and other user interface components are described in described in commonly assigned U.S. Patent Application Publication No. 2018/0361055, titled AUTOMATED FLUID MANAGEMENT SYSTEM, the entire disclosure of which is hereby incorporated by reference.

The touch screen interface 42 may be operatively connected to or a part of the controller 30. The controller 30 may be a CPU, including a computer, tablet computer, or other processing device. The controller 30 may be operatively connected to one or more system components such as, for example, an inflow pump, a fluid warming system, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The fluid flow rate or the fluid pressure of fluid provided by the fluid management system 10 at any given time may be displayed on the display screen 44 to allow the operating room (OR) visibility for any changes. If the OR personnel notice a change in fluid flow rate or fluid pressure that is either too high or too low, the user may manually adjust the fluid flow rate or the fluid pressure back to a preferred level. The fluid management system 10 may also monitor and automatically adjust the fluid flow rate or the fluid pressure based on previously set parameters.

In some embodiments, the fluid management unit may include one or more collection containers (not shown), for collecting waste fluid during a medical procedure. The collection containers (e.g., canisters) may be in fluid communication with a vacuum pump to provide suction for drawing fluid into the collection containers. The vacuum pump may be operatively and/or electronically connected to the controller 30. In some embodiments, the vacuum pump may be disposed within the fluid management system 10. Other configurations are also contemplated. In some embodiments, the collection container(s) may be operatively coupled to a collection load cell to detect placement and/or weight of fluid in the collection container(s) to contribute to a fluid deficit calculation.

The console 20 may include a door 50 hingedly attached to the housing 22 of the console 20. The door 50 may be opened to access a receptacle 52 configured to receive a fluid cassette of a single use fluid tubing set therein. The fluid management system 10 may include an inflow pump configured to operatively engage the fluid tubing set to pump and/or transfer fluid from the fluid supply source 33 (e.g., a fluid bag, etc.) through the fluid tubing set to a treatment site during a medical procedure. Some illustrative fluid cassettes are described in described in commonly assigned U.S. Patent Application Publication No. 2022/0370706, titled FLUID MANAGEMENT SYSTEM, the entire disclosure of which is hereby incorporated by reference.

An illustrative fluid management unit may include one or more fluid container supports, such as fluid supply source hanger(s) 32, each of which may support a fluid supply source (e.g., fluid bag) 33. In some embodiments, placement and/or weight of the fluid supply source(s) 33 hanging from the fluid supply source hanger(s) 32 may be detected using a remote sensor and/or a supply measurement load cell 31 associated with and/or operatively coupled to each fluid supply source hanger 32 and/or fluid container support for monitoring a state of a fluid supply source. For example, a remote sensor or supply measurement load cell 31 may be provided for each fluid supply source hanger 32. The controller 30 may be in electronic communication with the supply measurement load cell 31. The fluid supply source hanger(s) 32 may be configured to receive a variety of sizes of the first fluid supply source(s) 33 (see, for example, FIG. 2) such as, for example, 1 liter (L) to 5 L fluid bags (e.g., saline bags). It will be understood that any number of fluid supply sources 33 may be used. The fluid supply source hanger(s) 32 may extend from the housing 22 of the console 20 and may include one or more hooks 35 from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

The weight of the hung fluid supply source(s) 33, as measured by the measurement load cell 31, may be displayed on the touch screen interface 42 to inform the physician how much fluid they have left before they must change the fluid supply source 33. It is important that the amount displayed on the screen is accurate so the physician can accurately predict when they need to switch out the fluid supply source 33 and not waste time during a procedure. However, the manufacturing variability in load cells 31 may cause each measurement load cell 31 to have an offset of varying magnitude. The offset may be a DC voltage that is present in the output of the measurement load cell 31 even when no weight is applied to the measurement load cell 31. This inherent offset may negatively impact the measurement system because it introduces error, limits the workable gain, and decreases resolution. That is, a non-zero output of the load cell 31 could be corrected digitally by simply adding a fixed value to the reported measured value when the load cell 31 is unloaded, but that limits the available dynamic range of the measurement circuit, reducing gain and resolution.

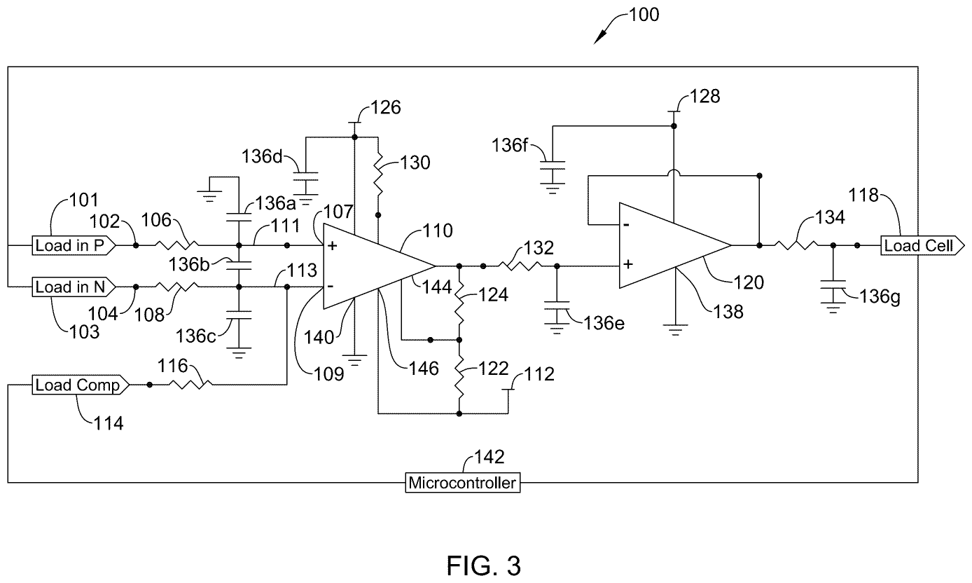

FIG. 3 is an illustrative circuit diagram 100 of a compensation circuit that may be electrically coupled with the circuitry of the measurement load cell 31 to minimize the offset of the measurement load cell 31. The fluid management system 10 may include a compensation circuit 100 for each measurement load cell 31. Thus, if the fluid management system 10 includes two load cells 31, the console 20 may house two compensation circuits each electrically coupled to a separate measurement load cell 31. While not explicitly shown, the fluid management system 10 may include control circuitry configured to facilitate the determination of an actual offset of a particular measurement load cell 31 and determine an appropriate offset voltage to apply. In some cases, the control circuitry may be incorporated into the controller 30. Alternatively, or additionally, the control circuitry may be incorporated into another controller. The controller may take many forms, including, for example, a microcontroller or microprocessor, coupled to a memory storing readable instructions for performing methods as described herein, as well as providing configuration of the controller for the various examples that follow. The controller may include one more application-specific integrated circuits (ASIC) to provide additional or specialized functionality, such as, without limitation a signal processing ASIC that can filter received signals from one or more sensors using digital filtering techniques. Logic circuitry, state machines, and discrete or integrated circuit components may be included as well. The skilled person will recognize many different hardware implementations are available for a controller.

In some cases, a measurement load cell 31 may have an offset that ranges from −15 millivolts per volt (mV/V) to +15 mV/V with a maximum full-scale span of 24 mV/V. In such an instance, the output may range from −15 mV/V to about 39 mV/V. The compensation circuit may reduce the output range of the example measurement load cell 31 to about 0 mV/V to about 25 mV/V. These are just illustrative ranges. The measurement load cell 31 may have other offset ranges. A measurement load cell 31 may, for example, use a Wheatstone bridge configuration in which four strain gauges placed in the arms of the bridge: R1, R2, R3 and R4, obtain an output signal proportional to the applied force. Other designs may be used, such as a capacitive load cell. Such devices are subject to offset for various known reasons, such as but not limited to minor mismatch of the components as well as mechanical or thermal stresses in and after manufacturing.

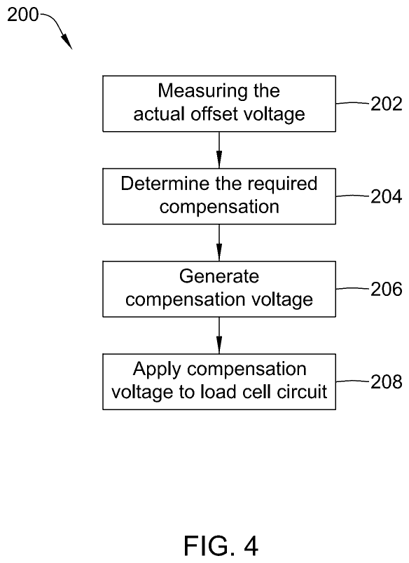

Referring additionally to FIG. 4, which illustrates a flow chart of an illustrative method 200 for determining an actual offset of the measurement load cell 31 and applying a corrective bias, the method 200 may begin by the controller 30 or control circuitry measuring the actual offset voltage of the measurement load cell 31, as shown at block 202. This may be performed during a calibration phase. In some examples, the calibration may be performed by a microcontroller 142 that is operably connected to the output of the load cell 118. The microcontroller 142 may include control circuitry and logic configured to perform the calibration. For example, the microcontroller 142 may include analog-to-digital converter (ADC) input pins with which it measures the output voltage from the circuits of the load cell 118. The microcontroller 142 may further include digital-to-analog converter (DAC) output pins where it can generate an output voltage (e.g., load compensation 114) controlled by the software within the microcontroller 142. For example, the user may initiate a calibration phase as the touch screen interface 42 with no weight applied to the measurement load cell 31 (e.g., the hanger 35 is free from a fluid source 33). Any voltage generated during the calibration phase (in the absence of an applied force) may be considered to be the offset voltage. During the calibration phase, the measurement load cell 31 may generate a differential voltage across a first measurement load cell output node 101 and a second measurement load cell output node 103 (see, for example, FIG. 3).

Measuring the measurement load cell offset with no weight applied to the hanger 35 is one way of performing calibration with the load cell 31 in a known state. A predetermined weight could be applied instead, for example to approximate any tare weight that the load cell 31 would typically or even always encounter, such as the weight of an empty fluid container or bag. In further examples, more than one calibration may be used to account for different fluid container types or sizes. Having more than one calibration may allow the calibration of the measurement load cell to be re-centered relative to the dynamic output range of the circuitry for a range of fluid container types, sizes, or tare weights.

The differential voltage may be an analog signal. The first measurement load cell output node 101 may be connected to a first input 102 of the compensation circuit 100 and the second measurement load cell output node 103 may be connected to a second input 104 of the compensation circuit 100. The first input 102 may be connected in series with a first resistor 106 to form a first input line 111 and the second input 104 may be connected in series with a second resistor 108 to form a second input line 113. The first and second resistors 106, 108 may provide input protection and help set the input impedance for an operational amplifier 110. The first and second resistors 106, 108 may help ensure proper signal condition of the differential voltage from the measurement load cell 31 before the signal is further processed. The first and second resistors 106, 108 may have a same resistance. The non-inverting input 107 of the first operational amplifier 110 may be coupled to the first line and an inverting input 109 of the first operational amplifier 110 may be coupled to the second input line 113. The first operational amplifier 110 may scale and measure the differential voltage between the input nodes of the operational amplifier 110. A load compensation is provided at 114, coupled to the inverting input in this example by resistor 116. The load compensation 114 could, if desired, instead be coupled to the non-inverting input 107 of the operational amplifier 110 in other examples. Load compensation 114 may be implemented as a digital to analog circuit to is used to limit the measurement offset, as further described below.

Next, the required compensation to bring the offset of the measurement load cell 31 into a desired range may be determined, as shown at block 204. For example, the required compensation to bring the offset of the measurement load cell 31 to +/−1 mV/V may be determined. The measurement of the differential voltage during the calibration phase may be used to tune an output node of the compensation circuit 100 for an individual measurement load cell 31. At the start of calibration, load compensation 114 is off, and the operational amplifier 110 may generate an output that is proportional to the offset of the particular measurement load cell 31. A unity gain operational amplifier 120 then provides the measurement output at 118 which, during calibration (zero load on the load cell and the load compensation 114 off) would equal the load offset. During calibration, in a no-load condition, the microcontroller 142 may run a function which will adjust the voltage at the load compensation 114 until the load cell 118 has the desired voltage, indicating that the load cell offset is balanced by the DAC voltage. The controller 30 or microcontroller 142 may use the offset voltage to tune a digital-to-analog converter (DAC) 114 to generate a controlled output. In some cases, an output 118 of the compensation circuit 100 may be monitored as the output of the DAC 114 is adjusted until the output 118 of the compensation circuit 100 is within a desired range. In some cases, the desired range may be from about-1 mV/V to about 1 mV/V. However, this is not required. The controller 30 or control microcontroller 142 may store the DAC 114 output which achieves the desired range as the required compensation.

Next, the compensation circuit 100 may generate the compensation voltage, as shown at block 206. For example, the controller 30 or microcontroller 142 may be configured to generate a compensation voltage using the DAC 114. The DAC 114 may supply a precise voltage to counteract the measured offset. The DAC 114 may be electrically coupled to the second input line 113. A current limiting resistor 116 may be connected in series with the DAC 114 output which may limit the current and ensure proper integration with the existing load cell circuit. The current limiting resistor 116 may have a resistance that is at least ten times the resistance of the first and/or second resistors 106, 108. Generally, the offset voltage may bias the circuit of the measurement load cell 31 in the opposite direction of the factory load cell offset.

Next, the compensation voltage may be applied to the circuit of the measurement load cell 31, as shown at block 208. For example, the controller 30 or microcontroller 142 may be configured apply the compensation voltage to the circuit of the measurement load cell 31. By applying the compensation voltage to the circuit of the measurement load cell 31, the system “tares” or “zeroes” out the inherent offset of the measurement load cell 31. This may result in a more accurate “zero” point for the measurements of the measurement load cell 31. It is contemplated that at least the measuring step 202 and the determining step 204 may occur under zero load conditions. Said differently, the measuring step 202 and the determining step 204 may occur before a fluid supply source 33 has been hung from the hook 35 or the fluid supply source hanger(s) 32. In some cases, the measuring step 202 and the determining step 204 may be repeated at predetermined intervals or set times (e.g., before a procedure, once a day, once a week, etc.) to account for any drift in the characteristics of the measurement load cell 31 or due to environmental factors (temperature, for example).

A third resistor 122 and a fourth resistor 124 may form a feedback network for the first operational amplifier 110 which sets the gain to 125. The feedback network may be electrically coupled to the operational amplifier 110 at an analog feedback input 144. The feedback may be for the reference voltage 112 of the operational amplifier 110 received at input 146. For example, the third resistor 122 may be a 100 Ohms resistor and the fourth resistor 124 may be a 12.4k Ohms resistor. The gain may be calculated using the formula of Equation 1:

$$Gain = 1 + ( 12 , 400 / 100 ) \qquad Equation 1$$

The gain of 125 may be approximately five times greater than the gain of an uncompensated measurement load cell 31. This may increase the resolution of the output of the measurement load cell 31 after the offset compensation has been applied relative to a measurement load cell 31 without compensation. This voltage is buffered at 120 with a unity gain operational amplifier, using associated circuits to smooth the resultant output voltage 118. Generally, the output voltage 118 may be calculated using the formula of Equation 2:

$${V}_{out} = ( {V}_{D} * Gain ) + {V}_{Offset} \qquad Equation 2$$

where Vₒᵤₜ is the output voltage 118, VD is the voltage differential across the first measurement load cell output node 102 and the second measurement load cell output node 104, and V_{Offset} is the determined voltage offset delivered by the DAC 114.

The control circuit 100 operational amplifiers are supplied by reference voltages 126, 128. The reference voltages 126, 128 may provide a stable reference voltage for the circuit 100 to ensure accurate measurements and signal conditioning. A first reference voltage 126 may be connected in series with a fifth resistor 130. The fifth resistor 130 may act as a pull-up or pull-down resistor to help stable the reference voltage. The fifth resistor 130 may have a resistance approximately five times greater than the resistance of the first and/or second resistors 106, 108. The first reference voltage 126 may be used to control the reference voltage for the first operational amplifier 110 while the second reference voltage 128 may be used to control the reference voltage for the second operational amplifier 120.

The control circuit 100 may further include a sixth resistor 132 connected between the output of the first operational amplifier 110 and the non-inverting input of the second operational amplifier 120. The sixth resistor 132 may form part of the signal path between the two amplification stages and may contribute to setting the overall gain or input impedance for the second stage. The sixth resistor 132 may have a resistance of approximately 1.5 to 1.75 times the resistance of the first and/or second resistors 106, 108. A seventh resistor 134 may be connected between the output of the second operational amplifier 120 and the voltage output 118. The seventh resistor 134 may have a resistance of approximately five times the resistance of the first and/or second resistors 106, 108.

The control circuit 100 may further include a plurality of capacitors 136a-g. Some capacitors 136a, 136c may be connected to the inputs of the first operational amplifier 110. These capacitors 136a, 136b may help filter high-frequency noise from the positive and negative load cell input signals 101, 103, improving the signal-to-noise ratio. Some capacitors 136d, 136f may be connected to the reference voltage lines. These capacitors 136d, 136f may function as decoupling capacitors to reduce noise and ensure a clean reference voltage. At least one capacitor 136c may be connected to the power supply 140 of the first operational amplifier 110 and/or the power supply 138 of the second operational amplifier 120. This capacitor may help stabilize the power supply by filtering out any high-frequency noise or transients, ensuring clean power for the operational amplifiers 110, 120. Separate capacitors may be provided for each power supply. Another capacitor 136e may be coupled to the connected to the voltage offset line. This capacitor 136e may help stabilize the compensation voltage and reduce any noise or fluctuations in the offset correction. Another capacitor 136g may be connected to the output of the second operational amplifier 120. This capacitor 136g may serve as a low-pass filter, smoothing the output signal and reducing any high-frequency noise introduced by the amplification process.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fluid management system, comprising:
a fluid bag;
a measurement load cell for monitoring a state of the fluid bag, the measurement load cell receiving an input voltage and generating a differential voltage across a first measurement load cell output node and a second measurement load cell output node;
a compensation circuit coupled to the measurement load cell having:
a first input having a first series resistor leading to a first input line;
a second input having a second series resistor, leading to a second input line;
a first operational amplifier coupled to the first input line and the second input line; and
a digital to analog converter (DAC) coupled by a current limiting resistor to the second input line;
a microcontroller electrically coupled to the measurement load cell and the DAC.

2. The fluid management system of claim 1, wherein the DAC is configured by the microcontroller to generate a controlled output, and the controlled output is calibrated to the measurement load cell.

3. The fluid management system of claim 2, wherein the controlled output is calibrated to the measurement load cell by providing an input voltage to the measurement load cell, monitoring an output of the compensation circuit, and adjusting the DAC until the output of the compensation circuit is in a desired range, and storing a setting for the DAC at which the output of the compensation circuit is in the desired range.

4. The fluid management system of claim 1, wherein a resistance of the first series resistor and a resistance of the second series resistor are approximately equal.

5. The fluid management system of claim 4, wherein a resistance of the current limiting resistor is at least 10 times the resistance of the first series resistor.

6. The fluid management system of claim 1, further comprising a second operational amplifier configured to receive an output voltage from the first operational amplifier.

7. The fluid management system of claim 1, further comprising a fourth resistor and a fifth resistor coupled to the first operational amplifier and configured to set a gain of the first operational amplifier.

8. A fluid management system, comprising:
a fluid bag;
a measurement load cell for monitoring a state of the fluid bag, the measurement load cell receiving an input voltage and generating a differential voltage across a first measurement load cell output node and a second measurement load cell output node;
a compensation circuit coupled to the measurement load cell having:
a first input having a first series resistor leading to a first input line;
a second input having a second series resistor, leading to a second input line;
a first operational amplifier coupled to the first input line and the second input line;
a digital to analog converter (DAC) coupled by a current limiting resistor to the second input line; and
a controller configured to:
measure an actual offset voltage of the measurement load cell during a calibration phase;
determine a required compensation to bring the offset of the measurement load cell into a desired range;
generate a compensation voltage using the DAC; and
apply the compensation voltage to the circuit of the measurement load cell.

9. The fluid management system of claim 8, wherein the controller is further configured to repeat the measuring and determining steps at predetermined intervals to thereby adjust the compensation voltage and account for drift in the characteristics of the measurement load cell.

10. The fluid management system of claim 8, wherein the desired range is from about −1 mV/V to about 1 mV/V.

11. The fluid management system of claim 8, wherein a resistance of the first series resistor and a resistance of the second series resistor are approximately equal.

12. The fluid management system of claim 11, wherein a resistance of the current limiting resistor is at least 10 times the resistance of the first series resistor.

13. The fluid management system of claim 8, further comprising a second operational amplifier configured to receive an output voltage from the first operational amplifier.

14. The fluid management system of claim 13, further comprising a third resistor and a fourth resistor coupled to the first operational amplifier and configured to set a gain of the first operational amplifier.

15. The fluid management system of claim 14, wherein the gain is approximately 125.

16. The fluid management system of claim 8, further comprising a plurality of capacitors configured to:
filter high-frequency noise from the load cell input signals;
stabilize power supplies for the operational amplifiers;
smooth the output signal; and
stabilize the compensation voltage.

17. The fluid management system of claim 8, further comprising a reference voltage source coupled to the first operational amplifier.

18. A method for compensating a measurement load cell in a fluid management system, comprising:
measuring an actual offset voltage of the measurement load cell during a calibration phase;
determining a required compensation to bring the offset of the measurement load cell into a desired range;
generating a compensation voltage using a digital to analog converter (DAC);
applying the compensation voltage to the circuit of the measurement load cell; and
amplifying the compensated measurement load cell signal using a second operational amplifier with a gain of approximately 125.

19. The method of claim 18, further comprising repeating the measuring and determining steps at predetermined intervals to account for drift in the characteristics of the measurement load cell.

20. The method of claim 18, wherein the desired range is from about-1 mV/V to about 1 mV/V.
